(19) United States
(12) Patent Application Publication
(10) Pub. No.: US 2004/0241736 A1
(43) Pub. Date: Dec. 2, 2004

(54) Analyte determinations

(76) Inventors: Shonn P. Hendee, Albuquerque, NM (US); Howland D. T. Jones, Edgewood, NM (US); Dashiell A. Birnkrant, Albuquerque, NM (US); Robert D. Johnson, Albuquerque, NM (US); Russell E. Abbink, Sandia Park, NM (US); Robert G. Messerschmidt, Corrales, NM (US)

Correspondence Address: V. Gerald Grafe, esq., General Counsel, InLight Solutions, Inc., 800 Bradbury SE, Albuquerque, NM, 87106, US

(21) Appl. No.: 10/850,646

(22) Filed: May 21, 2004

Related U.S. Application Data
(60) Provisional application No. 60/473,524, filed on May 27, 2003.
(60) Provisional application No. 60/472,349, filed on May 21, 2003.

(51) Int. Cl. C12Q 1/68; G06F 19/00; G01N 33/48; G01N 33/50; G01N 31/00

(52) U.S. Cl. 435/6; 702/19; 702/22

(57) ABSTRACT

A method and apparatus for determining an attribute of a sample from a spectrum of the sample. The invention comprises samplers and methods of sampling that provide controlled optical pathlengths through the sample, increasing the accuracy of the attribute determinations. The invention is applicable, for example, in determining analyte concentrations in biological samples, such as concentrations of analytes such as glucose in human blood.

### CROSS REFERENCES TO CO-PENDING APPLICATIONS

This application claims priority under 35 U.S.C §119 to U.S. Provisional Ser. No. 60/473,524, entitled “Analyte Determinations”, filed May 27, 2003, the disclosure of which is incorporated herein by reference, and to U.S. Provisional Ser. No. 60/472,349, entitled “Analyte Determinations”, filed May 21, 2003, the disclosure of which is incorporated herein by reference.

### TECHNICAL FIELD

This invention relates to methods and apparatus for monitoring analytes of biological fluids using infrared spectroscopy. In particular, this invention relates to an apparatus for optically interrogating a biological sample and determining analyte constituents.

### BACKGROUND OF THE INVENTION

The present invention is suitable for use with a patient-attached system that draws biological fluid from a patient for optical analysis of analytes on a continuous, near continuous, or intermittent basis. After optical analysis, the fluid can be returned to the patient's body, or can be permanently removed and discarded. Examples of such fluids include blood, urine, cerebral spinal fluid, and plasma. Examples of biological fluid analytes that can be measured include glucose, lactate, bicarbonate ion, hemoglobin, pH, albumin, total protein, cholesterol, alcohol, triglycerides, urea, and creatinine.

The use of infrared vibrational spectroscopy to quantitatively measure analyte concentrations in biological fluids generally requires that the optical path through the fluid be known. In certain cases, it is desirable to measure biological fluid analytes using existing tubing, such as a venous or arterial catheter, without diverting fluids to a separate sampling apparatus. However, such tubing can have variable or undetermined optical path characteristics, creating an unsuitable optical sampling configuration. Accordingly, there is a need for means for determining and/or controlling the optical path through the biological fluid, thus making it possible to accurately measure the analyte concentrations.

In situations where biological fluids can be diverted into or flowed through a separate “on-line” sampling vessel, it is also important to control the optical path. In this case, however, the optical path can be defined a priori. There is a need for sampling apparatuses that allow for controlling the optical path while minimizing biological fluid volume in the sampler.

### SUMMARY OF THE INVENTION

A method and apparatus for determining an attribute of a sample from a spectrum of the sample. The invention comprises samplers and methods of sampling that provide controlled optical pathlengths through the sample, increasing the accuracy of the attribute determinations. The invention is applicable, for example, in determining analyte concentrations in biological samples, such as concentrations of analytes such as glucose in human blood.

### DESCRIPTION OF THE DRAWINGS

The drawings form a part hereof, and should be reviewed in combination with the text, and are meant to illustrate embodiments and aspects of the present invention.

FIG. 1 is an illustration of an apparatus according to the present invention embedded “in line” with blood flowing from the patient and through the system in a continuous ex vivo loop.

FIG. 2 is a schematic illustration of a suitable optical sampling system, comprising an optical sampling apparatus that enables light interaction with the biological sample, a light source that can deliver light comprising a plurality of wavelengths to the optical sampling apparatus, a collector that collects light that has interacted with the sample, a spectrometer, and a processor.

FIG. 3 is an illustration of an apparatus according to the present invention embedded “in line” with blood flowing from the patient and into the system through an ex vivo line.

FIG. 4 is an illustration of a patient-detached embodiment of the present invention.

FIG. 5 is a schematic illustration of a converging-lens-based system with focal length matched to catheter or sampling vessel curvature for optimal conservation of optical path length.

FIG. 6 is a schematic illustration of a compression method (lens-based or fiber-based).

FIG. 7 is a schematic illustration of another embodiment of an optical sampler, using multiple optical fibers configured as transmitter/receiver pairs.

FIG. 8 is an illustration of an embodiment of a patient-detached optical sampling apparatus.

FIG. 9 is an illustration of an embodiment of an optical sampling apparatus for biological fluids.

FIG. 10 is a schematic illustration of an optical sampling system for transmitting light through a controlled fraction of the total sample volume.

FIG. 11 is a schematic illustration of a method for controlling optical path length by displacing sample.

FIG. 12 is a schematic illustration of an optically compatible sample displacer with a centrally contained sample volume.

FIG. 13 is a schematic illustration of an optically compatible sample displacer with optical power for refracting light rays in a controlled manner.

FIG. 14 is a schematic illustration of an apparatus for reflecting light through a controlled optical path.

### DESCRIPTION OF THE INVENTION

The following description describes illustrative embodiments and is not intended to limit the scope of the invention.

As used in this specification and the appended claims, the singularforms “a”, “an”, and “the” include plural referents unless the context clearly dictates otherwise. Thus, for example, reference to a method of classifying “a biological sample” includes a method of classifying more than one biological sample regardless of source. As used in this specification and the appended claims, the term “or” is generally employed in its sense including “and/or” unless the context clearly dictates otherwise.

FIG. 1 is an illustration of an apparatus according to the present invention embedded “in line” with blood flowing from the patient and through the system in a continuous ex vivo loop. Blood is drawn from the body through a catheter, flowing into the optical sampling system and then returned to the patient through a catheter. The present invention can be used to measure analytes of interest in biological fluids drawn directly from a patient's body in a system like that shown in FIG. 1. In this patient-attached embodiment, blood is drawn from the patient through a catheter and transported to the optical sampling system. In this embodiment, blood flows from the patient to the optical sampling system and back to the patient in a continuous loop.

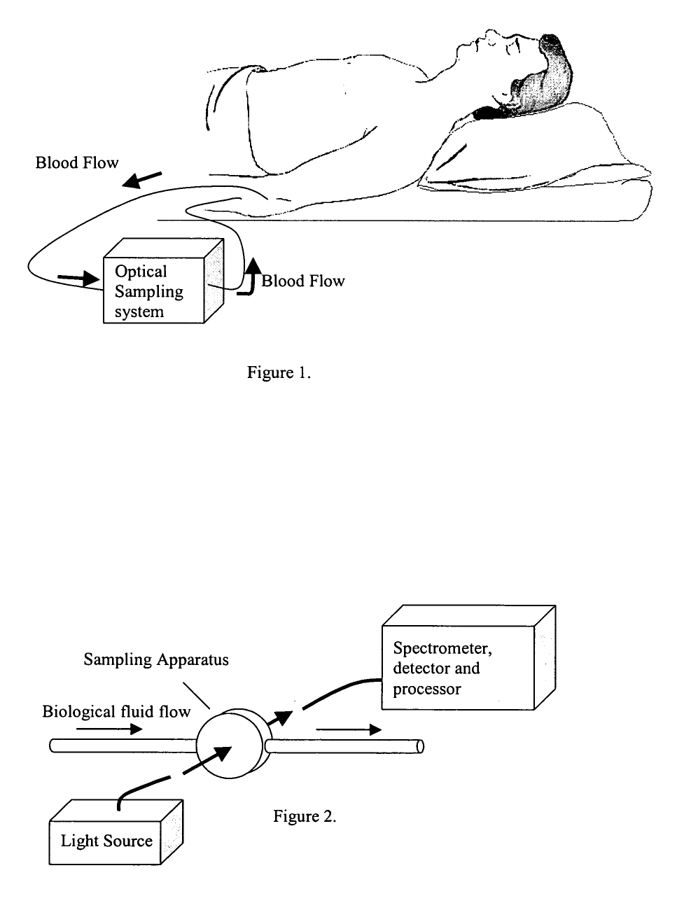

FIG. 2 is a schematic illustration of a suitable optical sampling system, comprising an optical sampling apparatus that enables light interaction with the biological sample, a light source that can deliver light comprising a plurality of wavelengths to the optical sampling apparatus, a collector that collects light that has interacted with the sample, a spectrometer, and a processor. The optical sampling apparatus affects the manner in which light is delivered to and collected from the biological sample. Details of several suitable sampling apparatuses are described below. Generally, however, a light source generates infrared light that is directed to the sampling apparatus, where the light interacts with the biological sample. The exiting light is directed to a spectrometer that yields an absorbance spectrum. Those skilled in the art will recognize that the spectrometer can be placed at various points in the optical path between the light source and the optical detector. Those skilled in the art will also recognize that the optical source can comprise a plurality of narrow wavelength devices, such as light-emitting diodes or laser diodes, for example. The optical measurement system processes the absorbance spectrum using a multivariate calibration model to yield measurements of attributes of the sample, e.g., constituents of a blood sample.

Additional instrument embodiments, examples of which are described below, utilize substantially the same method for optically determining attributes such as analyte concentrations, but differ in the manner in which the sample is presented to the instrument and/or handled at the conclusion of the optical measurement.

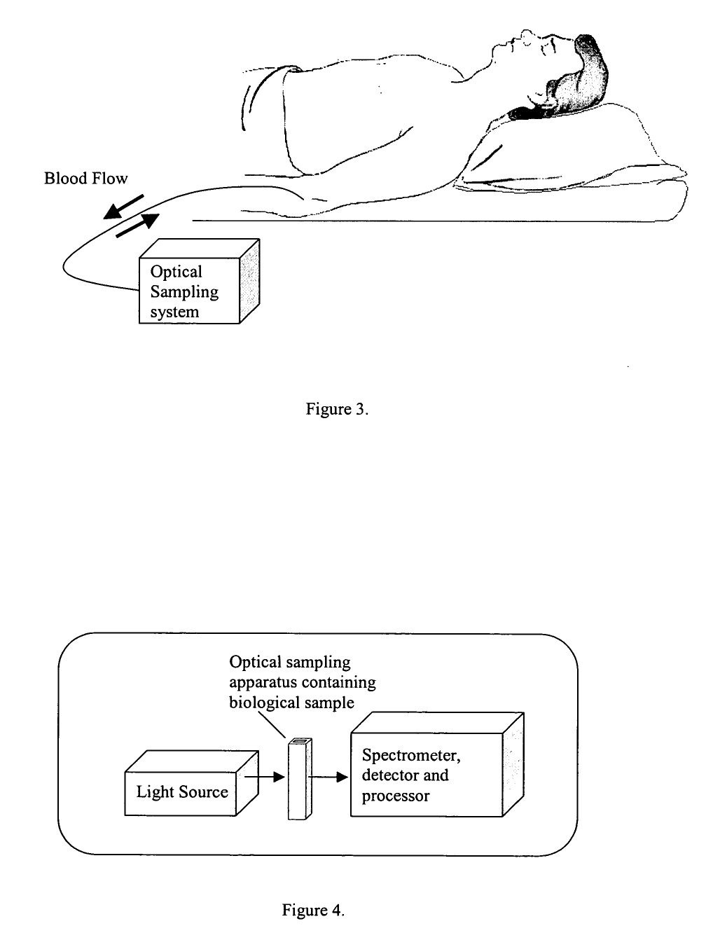

FIG. 3 is an illustration of an apparatus according to the present invention embedded “in line” with blood flowing from the patient and into the system through an ex vivo line. Blood is drawn from the body through a catheter, flowing into the optical sampling system and then returned to the patient through the catheter by reversing the flow direction. In the embodiment shown, blood is drawn into the sampling apparatus from the patient's body through a catheter. Analyte concentrations of the blood sample are measured with the optical measurement system, after which the blood is returned to the patient's body through the same catheter or tubing. The catheter is back-flushed, e.g., with saline or other fluid, to ensure that all blood is returned to the body. This system can also be used to measure alternative biological samples (e.g., urine or CSF). In such cases, it may be preferable to divert the sample to waste following the optical measurement.

In a patient-detached embodiment (FIG. 4), biological fluid samples are collected from the patient and placed in an optical sampling apparatus for analyte determination. The optical sampling apparatus containing the biological sample is placed in a sampling chamber in the instrument for optical determination of analyte concentrations. At the conclusion of the optical measurement, the sampling apparatus is removed to prepare the instrument for subsequent measurements.

Path Correction Methods when There is a Distribution of Optical Paths.

When there is a distribution of optical paths, correcting for that distribution using a correction method can improve accuracy of resulting analyte determinations. As an example, path length variation can be caused by optical scattering by the sample. As another example, path length variation can be caused by transmission through a system with nonuniform path length (such as the curved walls of a catheter). Path length correction refers to the use of algorithms to estimate and normalize or linearize the path length distribution through a sample. This approach has generally been applied to models of light interaction through tissue but can be useful for optically sampling a serum or other fluid sample contained in some non-ideal optical sampling geometry (such as cylindrical catheter tubing). The method combines the spectra of known absorbers in various concentrations and with varying path lengths through the sample to match the measured sample spectrum. This approach can be particularly useful in blood and serum samples, where the major absorbers/scatterers can be more constrained than in tissue. Path length distribution estimation methods according to the present invention can include direct parametric and nonparametric methods as well as pure simulation methods (for example, Monte Carlo methods based on the scattering and absorption properties of the system coupled with knowledge of the optical system properties and the sampling geometry).

Ratio of Concentration Methods

Two calibration models can be developed: an analyte model and a water model. These two models can be applied to spectral information from a sample of interest to accurately determine analyte concentration in the sample. The method utilizes two models: one to determine the analyte content in the spectrally interrogated space, and another to determine the water content in the space. The concentration of the analyte can be determined as the ratio of these two results. A nonlinear optimization algorithm can be applied to minimize the ratiometric prediction error associated with the combined measurements of each concentration (analytes and water), using a representative set of samples with known analyte and water concentrations as a calibration set.

Path Normalization Methods

Path correction can be achieved by calibrating the system based on a spectrally distinct reference analyte of known concentration. An example calibration approach comprises making both optical and reference measurements of one or more analytes in the biological fluid and determining appropriation corrections to the concomitant optical measurements. The correction can be applied to all prospective optical analyte determinations performed after the calibration, until a new path length calibration is performed.

Independent determination of path characteristics of the sample can enable adjustments to a spectral analyte determination that can improve accuracy. This can be particularly useful when there is variability in the mean path through the sample, which can be caused, for example, by: (1) variability in sample tubing diameter, and (2) variability in mean path due to positioning of the tubing in the sampler.

Spectral Identification of Outliers and Instrument Characteristics

Measurement of multiple analytes makes it possible to calculate a concentration-based Mahalanobis distance in addition to a spectral Mahalanobis distance. This can be useful for outlier detection. For example, the combination of results (incorporating not only the spectroscopic analyte determinations but also any other measurements made by the overall patient monitoring system) can be used to identify a corrupted or faulty sample characterized by a particular pattern of analyte results. Similarly, instrument problems can be identified based on the pattern of errors observed when a check-sample is run for quality control purposes. Correlations and relationships in the errors associated with different analytes can indicate specific problems with the instrument.

Sampler Considerations.

Specific fluid sampler designs can minimize variations of optical path through the sample. Sampler designs can also increase the signal to noise ratio (SNR) and can allow for measurement calibration and correction to compensate for variable instrument parameters (e.g., variation in tubing dimensions).

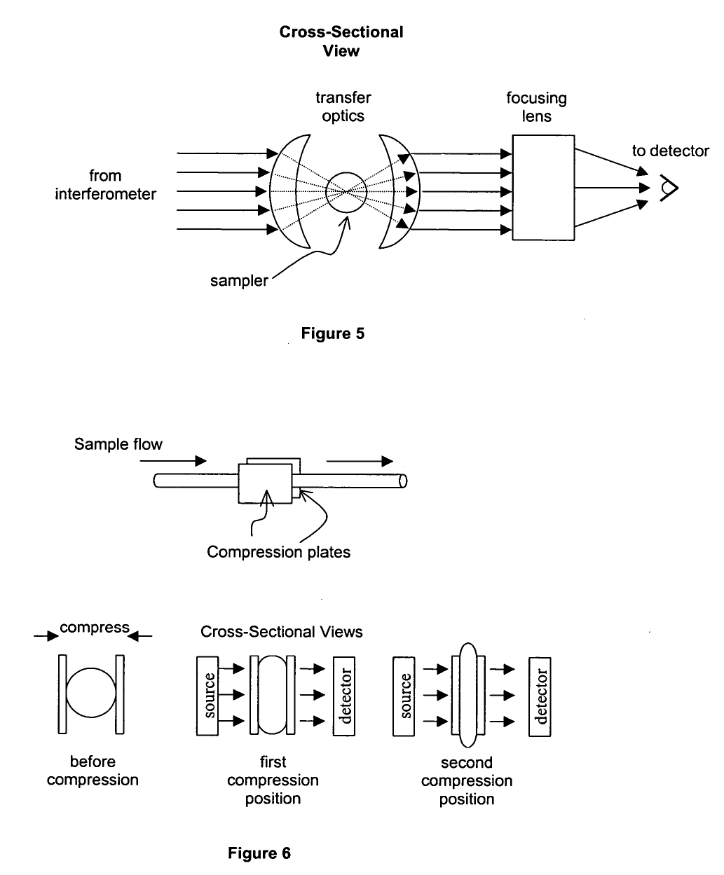

FIG. 5 is a schematic illustration of a converging-lens-based system with focal length matched to catheter or sampling vessel curvature for optimal conservation of optical path length. This optical sampling configuration minimizes path length variation through a cylindrical sampler such as a catheter by matching the focal length of a converging lens-based optical system in such a manner that the optical rays impend on the sampler surface with normal or near-normal incidence and focus at the center of the sampler.

FIG. 6 is a schematic illustration of a compression method (lens-based or fiber-based). Compressing a plastic sampler with round cross-section can be used to improve measurement accuracy by optimizing path length characteristics of the sampler. Compressing the sampler can: (a) minimize path length variation by creating nearly parallel surfaces through which the sample can be measured, (b) provide means for controlling the mean path through the sample such that the signal-to-noise ratio of the optical measurement can be optimized, and (c) provides means for making optical measurements at two or more path lengths to allow for differential optical determination of the analytes of interest for improved accuracy in the optical determination of the analytes (taking a differential measurement allows for the optical contributions of the sampler and instrument to be subtracted from the optical spectrum of the sample). By collecting data through the sample at two compression positions, it is possible to take the difference of the two spectra (both of which contain the spectral absorbance of the sample holder walls, and both of which contain a spectral representation of instrument state). The resulting spectrum is representative of the sample through the differential path length. A multivariate calibration model can be applied to the resulting spectrum to determine analyte concentrations.

In certain instrument configurations that make the optical measurement through existing fluid tubing, the optical path through the fluid sample can be excessively long to allow for adequate collection of photons at the detector. Thus it can be advantageous to reduce the path length through the fluid sample to optimize the spectral signal to noise ratio. Reduction of the optical path through sample can be achieved by compressing the tubing or other sample container. Furthermore, compressing the sample container can provide a flattened surface, thus advantageously reducing the variation in optical path length.

Due to the high water concentration in biological fluids, coupled with water's high optical density in the infrared region, the infrared absorbance spectrum obtained from a biological sample (e.g., blood, plasma, serum, cerebrospinal fluid and urine) is dominated by water absorption. Furthermore, water absorbs infrared radiation more strongly in some spectral regions than in others. For example, water absorbs infrared radiation much more strongly in the wavelength region from 4900 to 5500 cm⁻¹ than in the wavelength region from 6700 to 7300 cm⁻¹. To optimize the signal to noise ratio in collecting spectral data from a sample with highly variable optical density, it can be beneficial to collect data for the lower absorbance portion of the spectrum at one optical path through the sample, and then shorten the path (e.g., by compressing the sample tubing or vessel) to increase the SNR in the more highly absorbing region. The compression sequence, and corresponding optical path lengths, can be optimized for various analytes of interest, depending on the spectral characteristics of the sample and analytes.

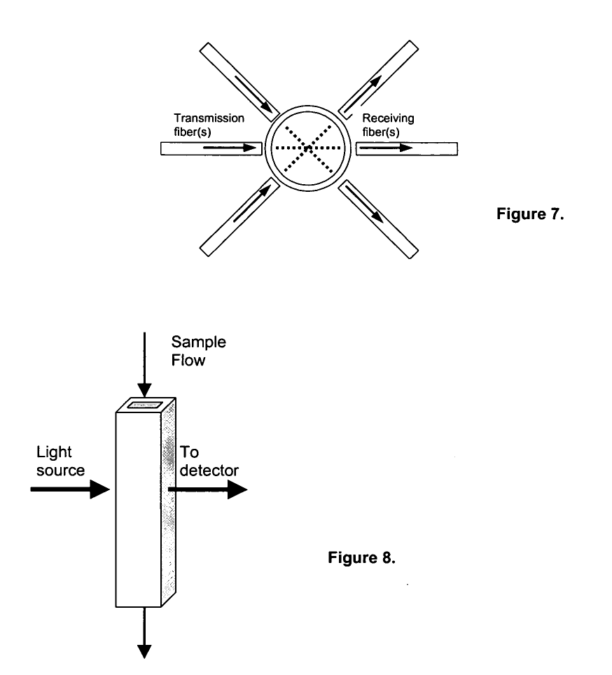

FIG. 7 is a schematic illustration of another embodiment of an optical sampler, using multiple optical fibers configured as transmitter/receiver pairs. In this sampler configuration, the transmitting fibers transmit light to their corresponding receiver fibers. The optical path length variation can be minimized by restricting the numerical aperture of the fibers, thus restricting the angular distribution of optical rays that pass through the sample to the receiving fiber.

Optical Sampling Apparatuses for In Vitro or Ex Vivo Analyte Measurement

For measurements in which the instrument is not patient-attached, but rather the sample is removed from the patient and transported to the instrument, it can be desirable to have a sampling apparatus that can be used to both collect the sample and hold the sample during the optical measurement. Several example embodiments of such an apparatus are described below. In each example, making the optical measurement involves steps of directing light into the optical sampling apparatus and collecting the resulting optical absorbance spectrum, and then applying a calibration model to the spectrum to determine the analyte concentration(s).

One embodiment of a patient-detached optical sampling apparatus comprises a hollow tube into which the biological fluid is drawn for optical measurement. The tube can be formed from suitable optical material (e.g., low-OH fused silica) for achieving acceptable throughput with minimal spectral interference. The material can also have parallel faces (rectangular) to minimize path variation. See as an example FIG. 8. It can also be advantageous to construct or form the sampler using low-cost optically compatible plastic or glass.

Another embodiment of an optical sampling apparatus for biological fluids comprises an optical probe that can be immersed in the sample to make an optical transmission measurement. This probe can be suitable for the situation where the sample is contained in a separate vessel or sample holder, and the probe is immersed in the sample to make the measurement. An example of such a transmission dip probe is shown in FIG. 9. This transmission probe can be used to make transmission measurements across a fluid-filled (for example blood or serum) gap. The source transmits across a fixed gap to a reflecting surface that deflects the light across the fluid-filled gap to another reflecting surface, which then directs the light to a receiving fiber. The gap distance and the position of the fibers relative to the reflecting surfaces define the optical path through the sample.

Sampler Considerations.

Sampler designs such as those below can minimize variations of optical path through the sample. Sampler designs such as those below can increase the signal to noise ratio (SNR) and can allow for measurement correction to compensate for variable instrument parameters (e.g., pipette tip variation).

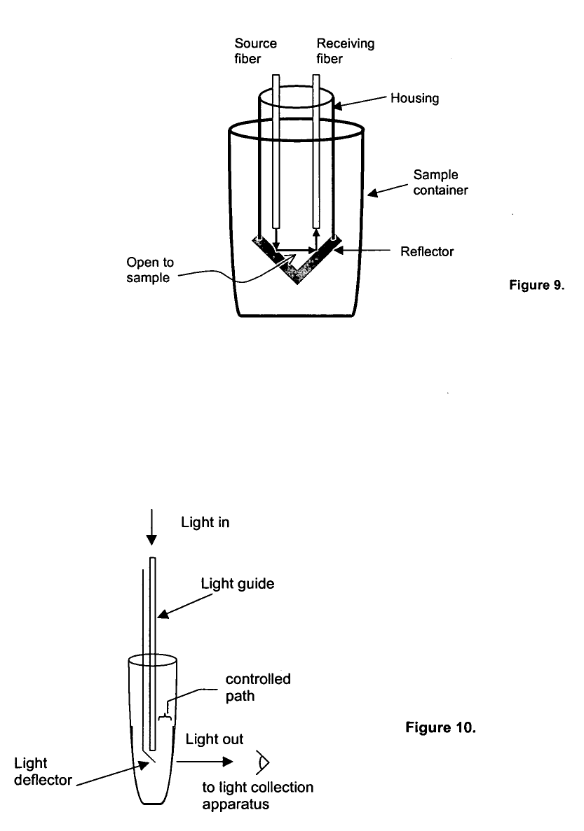

FIG. 10 is a schematic illustration of an optical sampling system for transmitting light through a controlled fraction of the total sample volume. An optical light guide can be inserted into the sample to control the optical path of the light that is transmitted through the sample. The light can be transmitted in a direct line with the light guide, or can be deflected such that it travels through a known path length of sample to a light collection apparatus (e.g., lens, light guide, or detector).

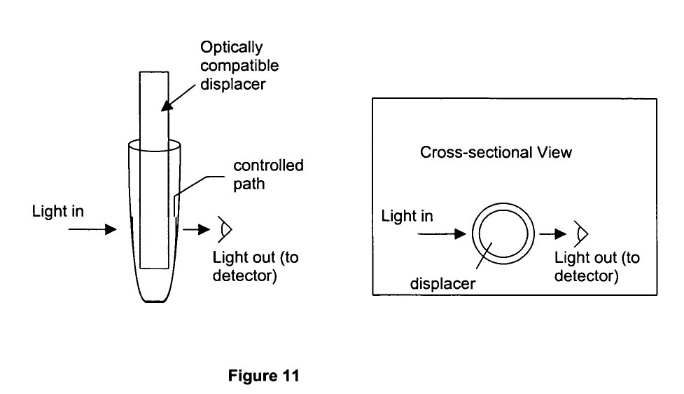

FIG. 11 is a schematic illustration of a method for controlling optical path length by displacing sample. The accuracy of analyte determinations of a sample contained in a vessel can be improved by volumetrically displacing the sample to control optical path and optimize signal-to-noise ratio. This apparatus can be comprised of material that has optical properties that are compatible with measuring the analytes of interest. The displacer can also comprise material or coatings that affect the optical interaction such that subsequent attribute determination is enhanced. As an example, the displacer can comprise a material that changes properties in relation to attributes of the sample, e.g., a dye embedded in a sol-gel. Phenyl red, for example, changes optical properties in relation to pH, so its incorporation into a displaced can allow optical determination of pH of a sample.

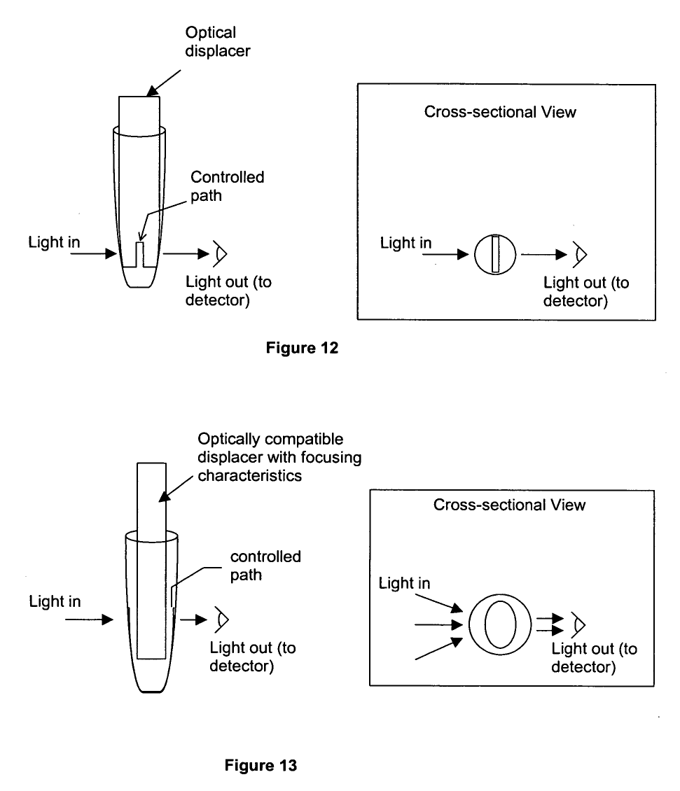

FIG. 12 is a schematic illustration of an optically compatible sample displacer with a centrally contained sample volume. Its operation is similar to the previous apparatus, except that the sample is contained within the center of the displacer rather than on the periphery. FIG. 13 is a schematic illustration of an optically compatible sample displacer with optical power for refracting light rays in a controlled manner. This is similar to the previous apparatus, except that the optical displacer has optical power for controlling the refraction of light rays to increase signal to noise ratio. An apparatus that has optical power (light focusing capability) would be capable of collecting light across a broader acceptance angle and focusing that light onto a light collection apparatus.

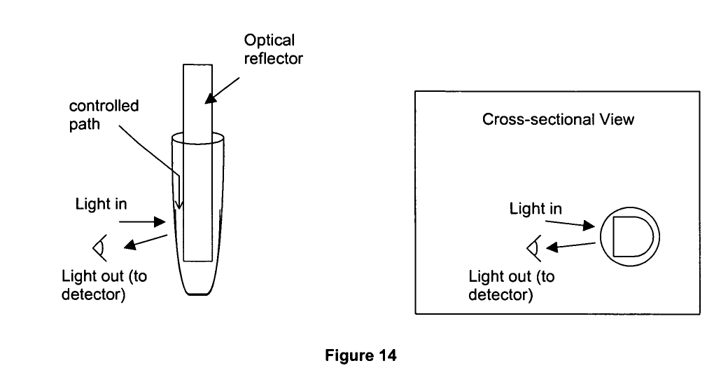

FIG. 14 is a schematic illustration of an apparatus for reflecting light through a controlled optical path. A reflecting device can be used to control the optical path through sample.

Determination of Sample Type and Suitability

The suitability of a sample for spectroscopic analysis as well as the type of sample presented for analysis can be determined and verified spectroscopically based on the optical characteristics of the sample. Examples are described below.

The spectral characteristics of a sample can be used to determine the sample type. This can be beneficial in ensuring the proper patient sample has been presented for analysis. For example, it is possible to distinguish a urine sample from a blood sample based on the sample absorption characteristics associated with hemoglobin and protein. Values that fall outside of a pre-defined pathophysiological range of hemoglobin and protein concentrations would trigger a warning or error message to the operator, indicating that there is likely a problem with the sample.

Biological specimens occasionally contain interfering substances that can adversely affect either the accuracy of the measurement or the interpretation of the results. For example, the process of acquiring a blood sample can occasionally cause lysis of the erythrocytes, which decreases the hematocrit of the sample. Interfering substances can also be associated with physiological effects, as is the case when a blood sample is drawn soon after a fatty meal and the sample has excessive lipid content. In such cases, it is useful to identify the interfering substance and, when possible, quantitate the substance. This information is useful in identifying problematic samples and providing important information regarding the suitability of the sample and the validity of the results. The presence and concentration of some interferents can be determined spectrally based on the absorbance characteristics of the interfering substance and on its concentration in the sample. Examples of interferents that can be spectrally determined include hemoglobin, bilirubin, and lipids. Interferents can also originate exogenously, as is the case in certain cardiovascular dyes (e.g., indo-cyanine green), imaging contrast agents, and some drugs of abuse (e.g., alcohol). In addition, the presence of interfering particles, such as clots or air bubbles, can be identified based on the spectral characteristics of the sample.

Analyte Determinations

The following are examples of analytes that can be suitable for NIR spectroscopic measurement in biological fluids (e.g., blood, serum, cerebrospinal fluid and/or urine). This list is not considered to be exhaustive.

| Total Protein | Albumin |
|---|---|
| Immunoglobulin G | Immunoglobulin M |
| Inmunoglobulin A | Microalbumin |
| Apolipoprotein B | Apolipoprotein Al |
| Complement Proteins 3 and 4 | Glycated Hemoglobin (HbAlc) |
| Haptoglobin | al-antitrypsin |
| Cholesterol | Bilirubin (total, unconjugated, |
| | conjugated) |
| Alcohol | Acetaminophen |
| Creatinine | C-Reactive Protein |
| Glucose | Cholesterol (total, HDL, LDL) |
| Phenytoin | Salicylate |
| Theophylline | Triglycerides |
| Valproic Acid | Vancomycin |
| Caffeine | Lipoprotein A |
| Blood Urea Nitrogen | Hemoglobin |
| Bicarbonate Ion | pH |
| Lactate | Triglycerides |

Example Instrument Timing

Acquisition of spectra over time can be informed by operation of the instrument. Examples of such considerations are described below.

The optical sampling time can be varied depending on which analytes are selected. There are differences in the signal-to-noise ratio among different analytes. Correspondingly, it may be important to optically sample some analytes for a longer period than others to meet the overall SNR required to meet accuracy requirements. The sampling time can be set according to the analyte selected.

Multiple Measurements

Multiple measurements can provide a health screener that can indicate and report whether a patient (based upon the optical measurement of the serum sample) is at high risk for a particular disease (for example, heart disease). This can be accomplished by measuring a panel of analytes that can characterize the disease state (agreed upon by physicians). Therefore, regardless of which analyte measurements are ordered, the system can always give a screening result. The ordering physician can then make a decision on whether additional tests are warranted. The ordering physician could ask for repeat or additional testing on a sample. In this way the initial test result triggers appropriate additional tests.

Instrument Applications

The spectroscopic system can be readily adapted to determine characteristics of the system itself, for example it can be readily adapted to determine characteristics of the sampler, including the blood tubing, for example, for use therewith. The spectral characteristics of the sampling vessel can be used to identify the sampling vessel material and to verify that its optical characteristics are compatible with the calibration model. For example, catheter material composition will vary according to manufacturer and catheter type. It is desirable that the instrument be able to identify the catheter and select the appropriate calibration as necessary.

New characteristics and advantages of the invention covered by this document have been set forth in the foregoing description. It will be understood, however, that this disclosure is, in many respects, only illustrative. Changes may be made in details, particularly in matters of shape, size, and arrangement of parts, without exceeding the scope of the invention. The scope of the invention is, of course, defined in the language in which the appended claims are expressed.

We claim:

1. A method of determining an attribute of a sample, comprising:
a. Determining a spectrum of the sample using a controlled pathlength; and
b. Determining the attribute from the spectrum.

2. A method as in claim 1, wherein determining a spectrum comprises:
a. Placing the sample in a carrier having a cross-section and defining a volume, and
b. Reducing the cross-section of the carrier as measured in a first direction;
c. Directing light through the sample along a second direction, where the second direction has a defined relationship to the first direction.

3. A method as in claim 2, wherein the defined relationship is substantially parallel.

4. A method as in claim 1, wherein determining a spectrum comprises:
a. Placing the sample in a carrier having a cross-section and defining a volume, and
b. Reducing the cross-section of the carrier a first amount as measured in a first direction;
c. Directing light through the sample along a second direction, where the second direction has a first defined relationship to the first direction, and determining a first spectrum of the sample from light collected;
d. Reducing the cross-section of the carrier a second amount as measured in a second direction;
e. Directing light through the sample along a third direction, where the third direction has a second defined relationship to the first direction, and determining a second spectrum of the sample from light collected;
f. Determining the attribute from a combination of the first and second spectra.

5. A method as in claim 4, wherein the second direction and the third direction are the same.

6. A method as in claim 1, wherein determining a spectrum comprises:
a. Placing the sample in a sample holder defining a volume;
b. Directing light along a plurality of paths, where each of the plurality of paths is characterized by a length through the sample, and where all such lengths are substantially equal;
c. Determining a spectrum from light collected.

7. A method as in claim 6, wherein
a. A portion of the sample holder defines a circular cross-section;
b. Directing light along a plurality of paths comprises directing light along paths radial with respect to the circular cross-section.

8. A method as in claim 1, wherein determining a spectrum comprises
a. Placing the sample in a sample holder;
b. Placing a probe having a controlled optical path therethrough in contact with the sample in the sample holder;
c. Determining a spectrum using the probe.

9. A method as in claim 8, wherein the probe comprises
a. A first lightguide,
b. A second lightguide spaced apart from and adapted to receive light from the first lightguide.

10. A method as in claim 1, wherein determining a spectrum comprises
a. Placing the sample in a sample holder defining a volume and having a cross-section;
b. Placing a sample displacer in the sample holder, where the sample displacer occupies a controlled portion of the cross-section;
c. Directing light through the sample holder and sample in the region of the cross-section and displacer along a path whose length is affected by the displacer;
d. Collecting light exiting the path and determining a spectrum of the sample therefrom.

11. A method as in claim 10, wherein the sample displacer comprises an optically transmissive material.

12. A method as in claim 10, wherein the sample displacer comprises an optically reflective material.

13. A method as in claim 10, wherein the sample displacer comprises an optically transmissive material shaped so as to direct light traversing therethrough.

14. An apparatus for determining an attribute of a sample, comprising:
a. A sample holder;
b. A sampling apparatus providing an optical path traversing the sample having a controlled optical pathlength;
c. An optical system that determines a spectrum of the sample from interaction of light with the sample along the optical path;
d. A processing system that determines the attribute from the spectrum.

15. An apparatus as in claim 14, wherein:
a. the sampling apparatus comprises means for reducing a cross-section of the sample holder along a first direction to a controlled length; and
b. the optical system directs light through the sample and sample holder along a second direction, where the second direction has a defined relationship to the first direction.

16. An apparatus as in claim 15, wherein the defined relationship is substantially parallel.

17. An apparatus as in claim 14, wherein:
a. the sampling apparatus comprises means for reducing a cross-section of the sample holder along a first direction to a first controlled length and to a second controlled length; and
b. the optical system directs light through the sample and sample holder along a second direction, where the second direction has a defined relationship to the first direction.

18. An apparatus as in claim 14, wherein:
a. The sampling apparatus comprises means for directing light through the sample holder and sample contained therein along a plurality of paths, where each of the plurality of paths is characterized by a length through the sample, and where all such lengths are substantially equal.

19. An apparatus as in claim 18, wherein
a. A portion of the sample holder defines a circular cross-section;
b. Directing light along a plurality of paths comprises directing light along paths radial with respect to the circular cross-section.

20. An apparatus as in claim 14, wherein the sampling apparatus comprises
a. A probe having a controlled optical path therethrough adapted to be placed in contact with the sample in the sample holder.

21. An apparatus as in claim 20, wherein the probe comprises
a. A first lightguide,
b. A second lightguide spaced apart from and adapted to receive light from the first lightguide.

22. An apparatus as in claim 14, wherein the sampling apparatus comprises
a. A sample displacer adapted to be placed in the sample holder, where the sample displacer occupies a controlled portion of the cross-section.

23. An apparatus as in claim 22, wherein the sample displacer comprises an optically transmissive material.

24. An apparatus as in claim 22, wherein the sample displacer comprises an optically reflective material.

25. An apparatus as in claim 22, wherein the sample displacer comprises an optically transmissive material shaped so as to direct light traversing therethrough.

26. An apparatus for determining an attribute of a patient's blood, comprising:
a. A recirculating blood loop, in fluid communication with the patient's blood;
b. An optical sampling system, in optical communication with the recirculating blood loop and adapted to determine a spectrum of blood in the recirculating blood loop;
c. A processing system that determines the attribute from the spectrum
